(19) United States
(12) Patent Application Publication
(10) Pub. No.: US 2010/0190181 A1
(43) Pub. Date: Jul. 29, 2010

(54) BIOMARKERS FOR PREECLAMPSIA

(76) Inventors: Julie Fisher, Yorkshire (GB); Elizabeth Turner, Yorkshire (GB); James Walker, Yorkshire (GB)

Correspondence Address: MYERS BIGEL SIBLEY & SAJOVEC, PO BOX 37428, RALEIGH, NC, 27627, US

(21) Appl. No.: 12/305,854

(22) PCT Filed: Jun. 26, 2007
(86) PCT No.: PCT/GB2007/050357

§ 371 Date: Mar. 24, 2010

(30) Foreign Application Priority Data
Jun. 27, 2006 (GB) 0612669.2

(51) Int. Cl. G01N 33/53; C12Q 1/02

(52) U.S. Cl. 435/7.1; 435/29

(57) ABSTRACT

The present invention relates to novel biomarkers such as histidine and ketone bodies and methods of use thereof for detecting preeclampsia in pregnant individuals and for identifying individuals at risk at an early stage of pregnancy.

The present invention relates to novel biomarkers and methods of use thereof for detecting preeclampsia in pregnant individuals and for identifying individuals at risk at an early stage of pregnancy.

### BACKGROUND

The pathogenesis of the human-specific pregnancy disorder preeclampsia (PE) is not fully understood. The disease develops in almost 10% of pregnancies, affecting both mother (by vascular dysfunction) and fetus (by intrauterine growth restriction), and can only be reversed by delivery of the baby, accounting for up to 15% of all preterm births. The syndrome normally manifests itself after 20 weeks of gestational age, presenting a high risk to both mother and baby, and is a major cause of maternal and foetal morbidity and mortality.

PE is characterised by the incidence of hypertension and proteinuria, as well as evidence of organ damage due to systemic vasoconstriction. Remission of these signs after delivery is the conclusive indicator of the disease. Hypertension is a result, not a cause of the disorder—the occurrence of both hypertension and proteinuria distinguishes preeclamptics from those suffering from gestational hypertension alone. Symptoms and signs can include headache, abdominal pain, decreased urine output, shortness of breath, hand and facial oedema, visual disturbances, confusion and apprehension, nausea and vomiting. Preeclampsia can also lead to seizures (eclampsia) in approximately 0.2% of pregnancies, and can be associated with complications such as coma and rarely maternal death.

Several aetiologies have been implicated in the development of PE, such as endothelial dysfunction, due to abnormal placentation or oxidative stress, due to the attack of reactive oxygenated species. Several indicators of endothelial dysfunction and oxidative stress have been proposed. For example, the superoxide dismutase enzyme (SOD) is the main defense against reactive oxygen species and so a decreased level of SOD in the blood has been used as a simple, sensitive marker for oxidative stress. Other markers proposed in protein-based assays include glutathione peroxidase, glutathione-S-transferase, nitic oxide synthase and exogenous compounds such as Vitamins C and E and β-carotene.

It is also known from the prior art to use low molecular weight systems as indicators of oxidation status. For example serum thiols, including amino acids such as cysteine and methionine are particularly susceptible to oxidation by ROS. The oxidative balance of glutathione, an endogenous antioxidant can establish the level of oxidative stress, by determining the ratio of the reduced form of glutathione, to the oxidised dimer, diglutathione. The levels of these in plasma have been found to be much lower in those women suffering from pregnancy-induced hypertension than those with a normal pregnancy.

Uncontrolled lipid peroxidation has also been associated with PE and it has been suggested that attack and conversion of polyunsaturated fatty acids, by ROS, into lipid hydroperoxides is the initial factor leading to vascular endothelial dysfunction in PE. Higher concentrations of the small molecular primary products of lipid peroxidation, like conjugated dienes (from rearrangement of lipid radicals), and stable secondary products, such as malondialdehyde and isoprostanes have been shown to be good markers of hypertensive diseases in pregnancy like PE. Malondialdehyde (MDA), a small, highly toxic molecule, is not only a marker of lipid peroxidation, but also has the potential to interact with DNA and be mutagenic. It has also been suggested that there is an interrelationship between malondialdehyde concentration (and therefore lipid peroxidation) and autoimmune response, both factors already individually correlated with severity of PE.

A number of metabolic products have been reported to correlate with the occurrence of preeclampsia, what is not known is the interrelation between these. Metabonomics has the potential to establish this, and at the same time to provide novel biomarkers for the condition which may ultimately lead to early diagnosis.

The present invention provides novel biomarkers for the specific prediction of preeclampsia in pregnant individuals.

### BRIEF SUMMARY OF THE DISCLOSURE

According to a first aspect of the invention there is provided a method of diagnosing preeclampsia in a pregnant individual or predicting the likelihood of developing preecampsia, the method comprising analysing a sample obtained from said individual for the presence of histidine or a methyl derivative thereof in said sample and comparing the levels to those values obtained for pregnant women at a similar gestational period and known not to be suffering from preeclampsia wherein an increased level of histidine or a methyl derivative thereof in the said sample indicates that the patient is suffering from or is likely to develop preeclampsia.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Preferably, the methyl derivative of histidine to be detected is 1 methylhistidine or 3 methyl histidine but more preferably is 1 methylhistidine as depicted in FIG. 15.

Preferably, the method includes the step of analysing a sample obtained from said individual for the presence of lipids in said sample and comparing the levels to those of values obtained for pregnant women at a similar gestational period and known not to be suffering from preeclampsia wherein a decreased level of lipids in the said sample indicates that the patient is suffering from or is likely to develop preeclampsia and/or;

Reference herein to a “biomarker” is intended to convey a molecular or chemical marker associated with a biological function. The biomarkers of the present invention can be used to indicate or measure a biological process specific to preeclampsia and can aid in the identification, diagnosis, and treatment of affected individuals and women who may be at risk but do not yet exhibit symptoms.

It will be appreciated that in the method of the invention the step of analysing a sample for increased levels of histidine or a methyl derivative thereof may be performed independently or in conjunction with step an analysis of lipid levels on the same or different sample obtained from the individual.

It is envisaged that a set of “normal” values will be established for normotensive pregnant women of varying gestational periods as a control set of values against which test samples may be compared. Accordingly, the control values will be derived from pregnant women know not to be suffering from preeclampsia and may be used as the standard against which tests can be compared.

Preferably, the individual is human but the method of the present invention may also be used on other mammals such as and without limitation apes, horses, cattle, dogs and cats or any other mammalian species which may suffer from preeclampsia.

Preferably, the sample is selected from the group comprising whole blood, blood plasma amniotic fluid and serum although other body fluids such as urine, synovial and cerebrospinal fluids may also be applicable.

Preferably, the sample may be fresh or frozen or otherwise preserved for subsequent testing.

Preferably, in the instance where the individual to be tested is a pregnant human the sample is taken between weeks 12 and the end of the gestation period and more preferably is taken at around 20 weeks gestation. The 20 week time period is the time at which preeclampsia may first becomes manifest and intervention is possible. However, it will be appreciated that the sample may be taken prior to this time period or after this date at a time when other routine maternal blood tests are carried out, for example and without limitation at the 12 week test or the 16 week “triple-test” stage or at the 30 week check up. Accordingly, the sample may be taken at a convenient time to coincide with the time at which routine investigations and monitoring take place.

It will be appreciated that the method of the present invention is performed ex vivo or in vitro from a sample obtained from the pregnant individual and that the sample may be fresh or stored.

Preferably, the method further includes the step of analysing the test sample for the presence of ketone bodies.

Preferably, the ketone body to be detected is selected from the group comprising 3hydroxybutyrate (3HB), acetoacetate (AA) or a ketoglutarate.

We have shown that blood plasma lipid levels decrease in pregnant women suffering from preeclampsia and also that some of the ketone bodies overlap at some points with lipid signals so that it is possible that both lipids and ketone bodies decrease in preeclampsia. The lipid marker of the present invention gives rise to signals at different locations, at one location there is no overlap with ketone bodies however at another location for the same lipid an overlap with decrease ketone bodies is observed.

Preferably, in one embodiment of the invention the sample is analysed by nuclear magnetic resonance (NMR) spectroscopy, and more preferably is ¹H-NMR spectroscopy.

In the method of the present invention, the effects of preeclampsia on metabolism have been examined using ¹H-NMR analysis of blood plasma. The use of chemometrics on this data has identified several biomarkers, the most striking being the changes in lipid and ketone body (3-hydroxybutyrate and acetoacetate) and histidine concentrations between the control group and the preeclamptic group. In each of the “bins” or regions of the spectrum where the variation across the study is most significant, a resonance assigned to a lipid or ketone bodies or histidine is located.

We have shown that corresponding loading plots, highlight the importance of lipids in this distinction. 3-hydroxybutyrate was identified in several of the influential regions of the spectrum, including δ 1.19-1.23 (bin 178), δ 2.36-2.40 (bin 153) and δ 4.09-4.17 (bins 115-6), the latter region also containing resonances, from lactate and proline. Other constituents playing a role in the division of the two groups were acetoacetate and valine, between δ 2.22-2.26 (bin 156).

Loading plots have shown consistently that the same sections of importance of the aromatic regions of the spectra which were influential in clustering of patients on the scores that histidine appeared to be the most important constituent. Two regions with the highest magnitude of eigenvectors, δ7.742-7.758 and 7.042-7.058, both correspond to signals assigned to histidine. Accordingly in one embodiment of the invention preeclampsia may be detected by this specific pattern of peaks.

It will be appreciated that the analysis and/or detection of the biomarkers of the invention may also be performed by techniques in the art other than NMR that are capable of measuring the levels of the biomarkers. For example, the levels of lipids and ketone bodies and histidine or a methyl derivative thereof in a sample may be measured by enzymic methods, antibody detection or separation techniques the selection of the analysis technique is not intended to limit the scope of the invention.

According to a second aspect of the invention there is provided use of histidine or a methyl derivative thereof in the detection of preeclampsia in a pregnant individual or in identifying an individual with an increased risk of developing preeclampsia.

According to a third aspect of the invention there is provided use of blood plasma lipid and histidine or a methyl derivative thereof in the detection of preeclampsia in a pregnant individual or in identifying an individual with an increased risk of developing preeclampsia.

Preferably, the second and third aspects of the invention includes any one or more of the features hereinbefore described with respect to the first aspect of the invention.

According to a fourth aspect of the invention there is a provided method of treatment of preeclampsia in a pregnant individual, the method comprising the steps of:
(i) detecting the presence of histidine or a methyl derivative thereof in said sample and comparing the levels to those of values obtained for pregnant women at a similar gestational period and known not to be suffering from preeclampsia wherein an increased level of histidine or a methyl derivative thereof in the said sample indicates that the individual is suffering from or is likely to develop preeclampsia;
(ii) detecting lipids in a sample obtained from said individual and comparing the levels to those of values obtained for pregnant women at a similar gestational period and known not to be suffering from preeclampsia wherein a decreased level of lipids in the said sample indicates that the individual is suffering from, or is likely to develop, preeclampsia; and
(iii) using the results obtained from step (i) and (ii) to determine a clinical treatment of the condition.

It will be appreciated that the order of performing steps (i) and (ii) may be reversed.

In the method of the fourth aspect of the invention, depending on the severity of the condition i.e. significantly decreased lipid compared to control values and/or significantly increased histidine or methylhistidine levels compared to control values the clinician may determine an immediate therapy or may delay the therapy and the results may also be aids to determine the aggressiveness of the therapy required.

Preferably, the fourth aspect of the invention includes the features hereinbefore described with respect to the first aspect of the invention.

According to a fifth aspect of the invention there is provided a method of identifying a pregnant individual at risk of developing preeclampsia, the method comprising analysing a sample obtained from said individual at around 15-25 weeks gestation period the method comprising:
(i) detecting the level of histidine or a methyl derivative thereof in said sample and comparing the levels to those of values obtained for pregnant women at a similar gestational period and known not to be suffering from preeclampsia wherein an increased level of histidine or a methyl derivative thereof in the said sample indicates that the individual is suffering from, or is likely to develop, preeclampsia; and
(ii) detecting the level of lipids in said sample and comparing the levels to those of values obtained for pregnant women at a similar gestational period and known not to be suffering from preeclampsia wherein a decreased level of lipids in the said sample indicates that the individual is suffering from, or is likely to develop, preeclampsia.

Preferably, the method further includes the step of repeating the analysis over subsequent weeks of gestation so as to monitor the progression of the disease or to monitor the efficacy of an intervening therapy administered to the individual to combat the condition.

Preferably, the fifth aspect of the invention includes the features hereinbefore described with respect to the first and third aspects of the invention.

According to a sixth aspect of the invention there is provided a diagnostic kit comprising reagents required to determine the level of the biomarkers of the present invention.

Preferably, the kit comprises any one or more features hereinbefore described according to any of the previous aspects of the invention.

### BRIEF DESCRIPTION OF THE DRAWINGS

FIG. 1A shows the expansions of the 1H-NMR spectra from women suffering from PE and FIG. 1B shows the expansions of the 1H-NMR spectra from normotensive pregnant women.

FIG. 2 shows score plots and loading line plots from PRINCIPLE component analysis (PCA) analysis of NMR metabonomic data. FIG. 2A shows scores of PRINCIPLE component 1 (PC1) vs PRINCIPLE component 2 (PC2) of plot data for study and control patients, FIG. 2B shows a loading plot corresponding to FIG. 2A and FIG. 2C shows a plot of the data after the region assigned to glucose was set to zero and FIG. 2D shows the loading plot corresponding to FIG. 2C.

FIG. 3 shows score plots and loading line plots from partial least squares discriminant analysis (PLS-DA) analysis of the same metabonomic data as FIG. 2A-D.

FIG. 4 shows a score plot of FIG. 2A.

FIG. 5 shows an expansion of the loadings line plot in (A) PCA analysis and (B) PLS-DA.

FIG. 6A shows PLS-DA scores plot and corresponding loading plot (FIG. 6B) of the data from the spin echo experiment, when “bins” of width 0.016 ppm were used, and data was normalised to unit total sum of the spectral integral. Influential regions of the spectrum, and the direction in which these occur, are shown on the loading plot (in ppm), and cause distinction between the women suffering from PE, those enjoying a normal pregnancy, women in their first pregnancy and women in their second or greater pregnancy.

FIG. 7 shows plots and loading scatter plots from PCA and PLS-DA analysis of the NMR metabonomic data, FIG. 7A shows PCA scores (PC1vs PC2), FIG. 7B shows a scatter plot loading corresponding to FIG. 7A, FIG. 7C shows PLS-DA scores (PC1 vs PC2) and FIG. 7D shows a scatter plot loading of FIG. 7C.

FIG. 8A shows the first increment of the NOESY sequence of a pregnant woman's plasma, FIG. 8B shows the aromatic region of a woman suffering from PE and FIG. 8C shows the same region of a normotensive pregnant woman. FIGS. 8 D, E and F correspond to the same groupings and regions but are taken from the CPMG spin echo experiment.

FIG. 9A-D shows PCA and PLS-DA score plots from the spin echo experiment and first increment NOESY pulse sequences when bins of width 0.045 ppm were used.

FIGS. 10 C and E shows PCA and FIGS. 10 A, B and D PLS-DA loading plots of the raw data from the spin echo experiment when bins of width 0.045 ppm were used.

FIGS. 11 A and B show PCA and FIGS. 11 C and D show PLS-DA scores plots of the raw data from the spin echo experiments when bins of width 0.016 ppm were used.

FIGS. 12 A and B show PCA and FIGS. 12 C and D show PLS-DA scores plots of the raw data from the spin echo experiments and first increment of the NOESY pulse sequence when bins of width 0.016 ppm were used.

FIG. 13A shows a PLS-DA score plot and corresponding load plot FIG. 13B of data from the spin echo experiments when bins of width 0.016 ppm were used and normalised to unit sum of spectral integral.

FIGS. 14A-D show score plots of the data from spin echo experiments and NOESY experiments where the data has been normalised indicating that normalisation is not appropriate.

FIG. 15 shows the chemical structure of histidine and its two methyl derivatives, 1 methylhistidine and 3 methylhistidine.

### DETAILED DESCRIPTION

Metabonomics is defined as the “quantitative measurement of the dynamic multiparametric metabolic response of living systems to pathological stimuli or genetic modification”, and has arisen from the application of ¹H-NMR spectroscopy to study the multicomponent metabolic composition of biofluids, cells and tissues. It can operate at all levels of an organism—organelle, cell, tissue, organ, or whole organism. In the present invention, the biological fluid to undergo analysis is the plasma of the pregnant woman, however other body fluids such as urine may also be appropriate.

NMR is especially suited for metabonomics and the study of biomarkers for PE, as it has the advantage of very little sample pre-treatment or preparation, is fast and non-destructive, and is also non-invasive, in that, although the blood to be analysed has to be taken from the pregnant woman, this is done routinely on admission into hospital.

Water is present in such high concentration in biofluids that its NMR peak is so huge that it can obscure other molecular information and cause dynamic range problems in the NMR detector. In addition, resonances of low molecular weight items of serum and plasma are very difficult to resolve as they are obscured by the broad envelope of high molecular weight resonances of plasma proteins, such as albumin.

NMR pulse sequences, like the first increment of the NOESY experiment, are therefore employed to suppress the water by selective irradiation of its resonance with a weak rf field during delays in the sequence. Spectral editing methods are applied, based on the spin properties of macromolecules (for example, plasma proteins) and small molecules (such as metabolites), spin echo loops like the Carr-Purcell-Meiboom-Gill (CPMG) pulse sequence can achieve this. Macromolecules have longer rotational correlation times and limited translational motion, and therefore have shorter T₁ and T₂ relaxation time constants and smaller translational diffusion coefficients than those of smaller molecules, allowing NMR spectra to be edited based upon these properties.

Overlapping signals of metabolites can also add to the complexity of the spectrum. The multicomponent nature of plasma means that its ¹H-NMR spectrum will not only be complicated, but ascertaining which peaks within a crowded region are those assigned to a possible biomarker of PE will be much more problematic. The aromatic region of the ¹H-NMR spectrum, from a chemical shift of approximately 6 ppm to 9.5 ppm, is an area with much fewer signals compared to further upfield, it is therefore a much less complex region, with fewer overlapping resonances, which will make possible biomarker identification much simpler.

After acquisition of all spectra, multivariate statistics can be applied to the study in a “chemometric” approach. Chemometrics is the term generally ascribed to Pattern Recognition (PR) and multivariate statistical approaches applied to chemical numerical data. As many samples are processed, automatic data reduction and PR analysis are needed this is achieved by dividing the NMR spectrum into regions of equal chemical shift ranges, followed by signal integration within those ranges (“binning”). By applying PRINCIPLE Component Analysis (PCA), the region of the spectrum with the most variation in intensity can be identified, allowing discrimination between control samples and preeclamptic patients, as well as identifying the possible biomarker that causes this variation. Partial Least Squares Discriminant Analysis (PLS-DA) can also be performed to improve the distinction made between the groups of pregnant women, by rotating PRINCIPLE components to achieve maximum separation and easier identification of the potential biomarkers.

### Materials and Methods

### Patient Selection

The pregnant women chosen for the study were all attending antenatal clinics, patients used as control subjects were normotensive pregnant women of similar gestation, whilst those women who were classed as suffering from preeclampsia were diagnosed according to the criteria of American College of Obstetrics and Gynecologists (ACOG) i.e. a rise in blood pressure after 20 weeks gestation to >140/90 mmHg on two or more occasions 6 h apart in a previously normotensive woman, combined with proteinuria. Proteinuria was defined as protein dipstick >1+ on two or more midstream urine samples, or a 24 h urine excretion of >0.3 g protein, in the absence of a urine infection. Severe preeclampsia was diagnosed if a blood pressure of >160/110 mmHg was observed. The women were selected in two stages those who were pregnant with their first child, and then those women who had already given birth to one or more children.

### Sample Preparation

Venous blood was collected in heparinised (lithium salt) anticoagulant tubes before centrifugation at 900 g for 10 minutes. The plasma was removed from the sample, and stored at −80° C. until required. The plasma was defrosted by warmth of hands/left at room temperature, before centrifugation at 900 g for 1 minute. A 0.17% w/v solution of the sodium salt of 3-(trimethylsilyl)propionic-2,2,3,3-d₄ acid (TSP) (Sigma-Aldrich, UK) in deuterium oxide (D₂O) (Fluorchem, UK) was prepared. 350 μl of this was added to 300 μl of the plasma in an eppendorf tube. The mixture was inverted three times before transferring all 650 μl to a 5 mm NMR tube (528PP-WILMAD, Sigma-Aldrich, UK).

### NMR Spectroscopy

¹H-NMR spectra were measured at 499.97 MHz on a Varian Unity Inova 500 spectrometer, and at 21° C. The Carr-Purcell-Meiboom-Gill (CPMG) spin echo pulse sequence [RD−90°−180°−τ)ₙ−acq] was used to suppress signals from macromolecules and other substances with short T₂ values. Relation delay (RD) represents a relaxation delay of 10.5 s, during which the water resonance is selectively irradiated. The spin-spin relaxation delay, 2 nτ, of 450 ms was used for all samples. As well as the spin echo experiment, the first increment of the NOESY pulse sequence [RD−90°−t₁−90°−tₘ−90°−acq] was employed to suppress the water peak, with irradiation of the water frequency during the recycle delay RD (9.93 s) and mixing time tₘ (150 ms). t₁ was set to 4 μs. For both the spin echo experiment and the first increment of the NOESY experiment, 512 transients were collected into 32,768 data points for each spectrum, with a spectral width of 8389.3 Hz.

### Data Analysis, Chemometrics and Statistical Siginificance

An exponential line broadening of 0.5 Hz was applied to each free induction decay (FIDs), prior to zero filling to 65,536 points, followed by Fourier transformation. Resultant spectra were phased and baseline corrected using VnmrJ 1.1D (Varian Inc., Palo Alto, Calif., USA). The spectra were “binned” into 224 segments, each with a width of 0.045 ppm, over the range δ −0.5-9.5, setting the range of δ 4.48-5.97 to zero, to remove the effects of varying efficiency in water suppression. The spectra were normalised relative to TSP, followed by a second analysis with normalisation to unit total sum of the spectral integral.

PRINCIPLE component analysis (PCA) and partial least square discriminant analysis (PLS-DA) were performed on mean-centred Pareto-scaled data, using SIMCA-P+ 11 (Umetrics, Umeå, Sweden). Visualisation of data was possible in the form of PC scores (t) plots, where each point on the scores plot is a sample in the study, revealing outliers and clustering. Loading plots (p) display each NMR spectral “bin”, and highlight the impact of these input variables on the scores (t), by exposing the spectral regions which are responsible for the positions of the samples, and therefore possible clustering, in the corresponding scores plots.

This procedure was also repeated, with the additional range of δ 3.20-3.94 set to zero, in an attempt to verify that glucose concentration in the plasma was not responsible for any clustering in the data.

After tests of normality had been performed, comparison of mean values of absolute integrals from dominant regions of the spectrum were carried out, between the PE group and control group, using the t-test or Mann-Whitney test, in the software SPSS 13.0 (SPSS Inc. Chicago, Ill., USA). P values were adjusted for multiple comparisons using false discovery rate in the software R2.4.1 (R Foundation for Statistical Computing, Vienna, Austria) and p values of less than 0.05 were regarded as significant.

### EXAMPLE 1

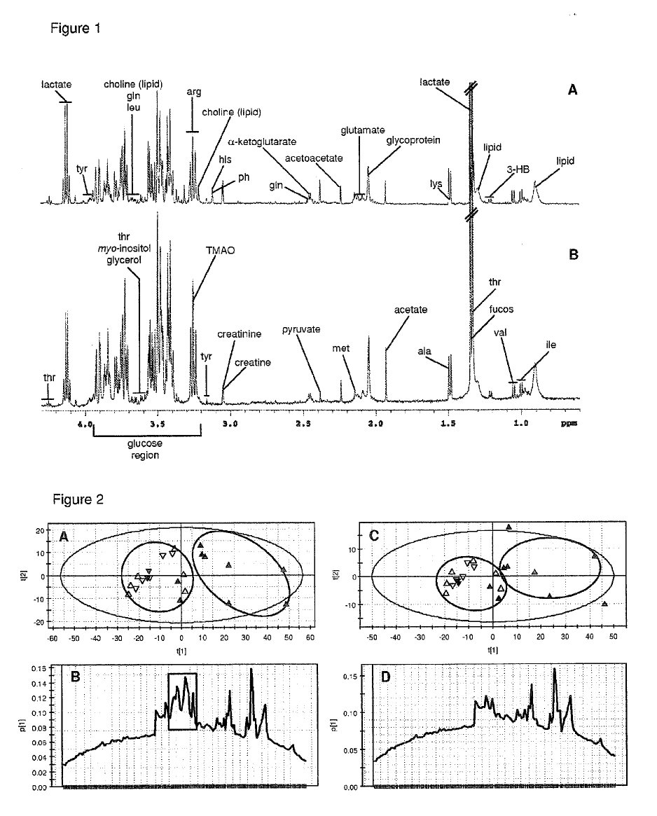

With reference to FIG. 1 there is shown expansions of the ¹H-NMR spectrum of the plasma of a women (A) suffering from PE; and (B) enjoying a normal pregnancy. (Abbreviations: 3-HB, 3-hydroxybutyrate; ala, alanine; arg, arginine; gin, glutamine; ile, isoleucine; leu, leucine; lys, lysine; met, methionine; thr, threonine; TMAO, trimethyl-amine N-oxide; tyr, tyrosine; val, valine.) Although the plasma ¹H-NMR spectra of the preeclamptic women ostensibly appeared similar to those of the control pregnant women (FIG. 1), PCA analysis clearly separated the two groups, with separation across both PCs 1 and 2 (FIG. 2). FIG. 2 shows scores plots and loading p[1] line plots from the PCA analysis of the NMR metabonomic data: FIG. 2 A shows scores (PC1 vs PC2) plot of the data, wherein open triangles represent women suffering from PE, and filled triangles represent women experiencing normal pregnancy, PCs 1 and 2 account for 92% of the data variance. FIG. 2 B shows loading plot corresponding to the scores in FIG. 2A, showing the most significant regions of the spectrum responsible for discrimination between the two groups of women, and the region assigned to glucose resonances (boxed); FIG. 2C scores (PC1 vs PC2) plot of the data after the integrals of the region assigned to glucose, δ 3.20-3.94, were set to zero. PCs 1 and 2 account for 94% of the data variance; FIG. 2D shows a loading plot corresponding to the scores in FIG. 2C. Triangles represent women who were pregnant with their first child, and inverted triangles represent pregnant women who had already given birth to one or more children. As expected, whilst the samples from women suffering from PE are quite clustered, a much greater within-group variability is observed for the control samples.

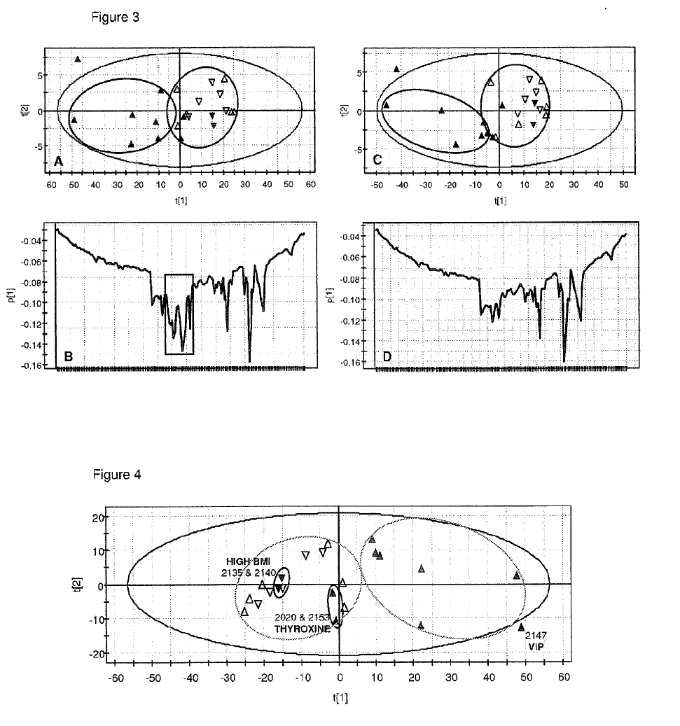

This separation between the two group of women was confirmed using PLS-DA. FIG. 3 shows scores plots and loading p[1] line plots from the PLS-DA analysis of the same NMR metabonomic data, confirming the results found in the PCA analysis. FIG. 3A shows scores (PC1 vs PC2) plot of the data, again, where open triangles represent those women suffering from PE, and filled triangles represent those women experiencing normal pregnancy. PCs 1 & 2 account for 84% of the data variance; FIG. 3B shows a loading plot corresponding to the scores in FIG. 3A, showing the most significant regions of the spectrum responsible for discrimination between the two groups of women, and the region assigned to glucose resonances (boxed); FIG. 3C shows scores (PC1 vs PC2) plot of the data after the integrals of the region assigned to glucose, δ 3.20-3.94, that were set to zero. PCs 1 & 2 account for 88% of the data variance and FIG. 3D shows a loading plot corresponding to the scores in FIG. 3C.

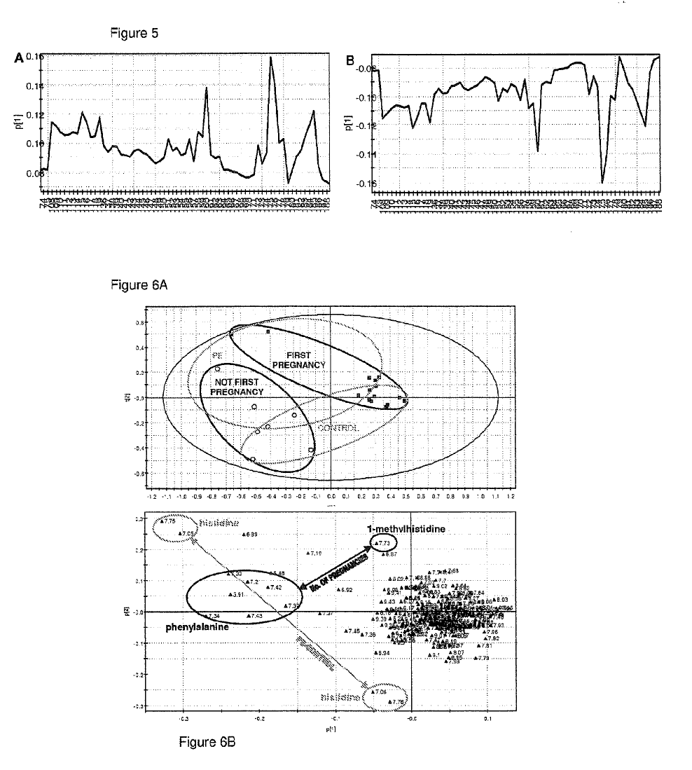

Some control outliers are present in the data, upon verifying medical history of the patients, samples 2135 and 2140, clearly together on the PC scores plot, are from women with a very high body mass index (BMI). Other control outliers, samples 2153 and 2020, are both from women who were prescribed the drug thyroxine. One control sample is clearly separate from all points in the analysis. This patient had taken part in a “Vitamins in Pregnancy” trial. FIG. 4 shows scores plot, from FIG. 2A, showing the control women who are outliers in the model and the apparent reasons for this exceptional behaviour.

Interrogation of the eigenvectors, shown in the loadings p[1] line plots of both the PCA and PLS-DA analysis, identified which spectral regions contributed most to the separation of classes. As can be seen in FIGS. 2 and 3, some of the conspicuous regions can be attributed to glucose. The level of glucose in the plasma is a parameter that is impossible to control, as some women were later found to have fasted, whilst others had not. Removal of the region assigned to glucose in the PCA analysis was achieved by setting the integration within those “bins” to zero, and re-performing the statistical analysis to verify that separation of groups still occurred. FIGS. 2 and 3 clearly show the same separation of groups, after “removal” of glucose, with tighter clustering of the preeclamptics, and with the same outliers present, confirming that glucose levels are not an indicator of health of pregnancy. The most significant eigenvectors in the p[1] and the loadings plots are still undoubtedly the same, before and after this intervention.

Initially only women experiencing their first pregnancy were included. Subsequently women on their second, third, or more, pregnancy were added. The discrimination was the same but the “tightness” of the clustering of these two subgroups differed. FIG. 5 shows expansion of the loadings p[1] line plot in (A) PCA analysis and (B) PLS-DA, highlighting the regions of the spectrum which are responsible for the distinction between the two groups of pregnant women, both analyses show the same significant regions of spectrum.

### EXAMPLE 2

FIG. 6 shows plots of the integral values obtained for the PE group (represented by open triangles) and control group of women (represented by filled triangles) for the most significant regions or “bins” of the NMR spectrum as these are assigned to lipids, the lower integral values of the preeclamptics validate the proposal of lipid peroxidation in PE. Our results show that differing lipid levels in the plasma of women suffering from PE, and those with normal pregnancy, are the main cause of the distinction between the two groups. Plots of the integral values obtained for each sample, shown in FIG. 6, indicate that the ranges of values (and hence concentration ranges) for PE samples are below the ranges obtained for the control samples, for all regions of the spectrum where a lipid resonance is observed.

### EXAMPLE 3

The spectral regions contributing most to separation were found to be in the chemical shift range δ 1.29-1.42 (bins 174-6). This is the region where signals from different types of lipid, although mainly VLDL lipids, lactate, fucose and threonine appear. The integral values of the PE group were found to be significantly lower than those of the control group (p=0.046). The second most influential region, δ 0.87-0.96 (bins 184-5), also involves lipids (including VLDL), cholesterol, and isoleucine and leucine, again, the integral values from PE samples were much lower than those of the control group, although not statistically significant (p=0.075). Region δ 2.22-2.26 (bin 156) is another significant reporter of the importance of lipids (p=0.018).

Glycoprotein and proline, both with resonances in the region of δ 2.04-2.08 (bin 160) were revealed as important constituents. 3-hydroxybutyrate was identified in several of the influential regions of the spectrum, including δ 1.19-1.23 (bin 178), δ 2.36-2.40 (bin 153) and δ 4.09-4.17 (bins 115-6), the latter region also containing resonances, from lactate and proline. Other constituents playing a role in the division of the two groups were acetoacetate and valine, between δ 2.22-2.26 (bin 156). Some dominant regions revealed on the p[1] plot of the eigenvectors corresponded to regions of the spectrum assigned to various amino acids, such as tyrosine, histidine and phenylalanine (δ 3.94-3.99, bin 119), methionine (δ 2.13-2.17, bin 158), glutamine (δ 2.08-2.13, bin 159), lysine (δ 1.47-1.51 and δ 1.89-1.93, bins 172 and 163 respectively) and arginine (δ 1.89-1.93, bin 163), as well as other components such as glutamate (δ 2.13-2.17 and δ 2.36-2.40, bins 158 and 153 respectively), α-ketoglutarate (δ 2.45-2.49, bin 151) and acetate (δ 1.89-1.93, bin 163).

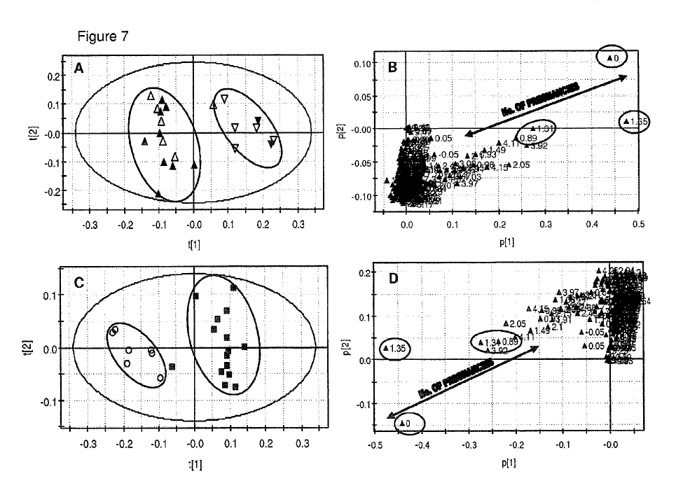

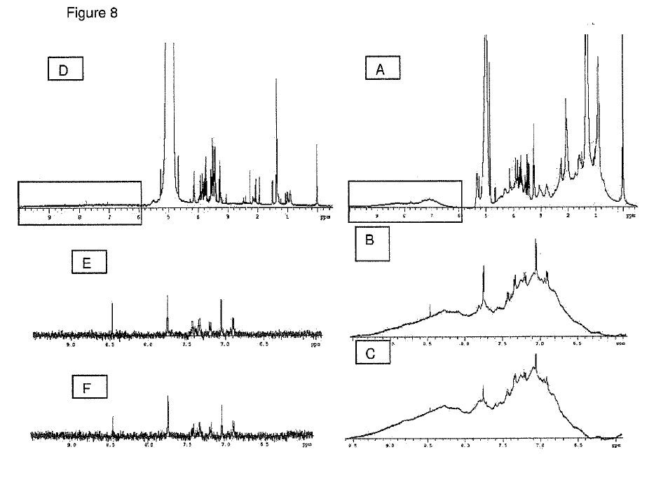

In the second analysis, where the integrals of the spectra were normalised to unit total sum of the spectral integral, PCA was able to distinguish between those women who were pregnant for the first time, and those women who were pregnant for the second (or more) time. FIG. 7 shows scores plots and loading scatter plots from the PCA and PLS-DA analysis of the NMR metabonomic data when integrals have been normalised to unit total sum of the spectral integral. FIG. 7 A shows PCA scores (PC1 vs PC2) plot of the data where open triangles represent women suffering from PE, and filled triangles represent women experiencing normal pregnancy. Triangles represent women in their first pregnancy, whilst inverted triangles represent women who have been pregnant before. PCs 1 & 2 account for 82% of the data variance; FIG. 7B shows loading scatter plot corresponding to the scores in FIG. 7A, showing the most significant regions of the spectrum responsible for discrimination between the women in their first pregnancy or their second or greater pregnancy; FIG. 7C shows PLS-DA scores (PC1 vs PC2) plot of the same data, where filled squares represent women in their first pregnancy and open circles represent those women who have been pregnant before. PCs 1 & 2 account for 80% of the data variance; FIG. 7D shows loading scatter plot corresponding to the scores in FIG. 7C.

Both PCA and PLS-DA (FIG. 7) were able to produce excellent separation between these two sub-groups of women, regardless of their health almost all preeclamptic and control women were correctly classified on the scores plots, with just one women (suffering from PE), who was pregnant with her first child, lying nearer to the group of women who were not pregnant for the first time.

The corresponding loading plots, again, highlight the importance of lipids in this distinction; the spectral regions contributing most to separation were found to be in the chemical shift ranges δ 0.87-0.91 (bin 185: lipid and cholesterol), δ 1.29-1.33 (bin 176: VLDL lipid, isoleucine, other lipids and fucose) and, most importantly, δ 1.33-1.37 (bin 175: lipid, threonine and lactate). All three of these regions exhibited significantly lower intensities, and therefore lipid concentration, in the plasma of those women pregnant for the first time than those who were not (p=0.004, p<0.001 and p<0.001 respectively).

In the first analysis, where normalisation was performed to the TSP resonance, classification of data points did not seem to depend on the number of children to which the women had given birth, although the pregnant women suffering from PE, who had given birth to one or more children, did seem to be grouped more tightly, especially after the removal of glucose from the statistical analysis, than those preeclamptic women who were pregnant for the first time. Normalisation to unit total sum of the spectral integral, however, allowed distinction between women who had had only one pregnancy and those women who had had more than one. Lipid concentration was, again, the discriminating factor between the two groups. However, as the level of the reference compound TSP, at 0 ppm, also appears to be changing (FIG. 7), it is also possible that the concentrations of plasma proteins in women pregnant with their first child are higher than those of the women not pregnant for the first time. As TSP binds to albumin this could account for the apparent reduction in intensity for this resonance observed in the spectra of women pregnant for the first time (p<0.001). As the number of pregnancies was not the aim of the investigation, the initial normalisation of integrals to the TSP resonance proved to be the best way to analyse our results to ensure a fair evaluation of the concentrations of constituents in the study of the pathogenesis of PE.

In summary, ¹H NMR analysis of the metabolic profile of plasma samples from pregnant women has enabled discrimination between healthy participants and those suffering with preeclampsia. Examination of the key components in the discrimination has identified a range of molecules all of which report on increased oxidation when preeclampsia is present. Many of the molecules arise from related metabolic events, detailed analysis of which may lead to a better understanding of the cause and pathogenesis of this disease.

### EXAMPLE 4

In a further experiment, the aromatic region of the ¹H-NMR spectra of both the preeclamptic women's plasma and that of the control group to all appearances looked very similar (FIG. 8). FIG. 8A shows a 1 CPMG spin echo (left) and first increment of the NOESY pulse sequence (right) spectra of a pregnant woman's plasma. The aromatic region from the spin echo spectrum appears to be very similar for the women suffering from PE (FIG. 8B left) and normotensive pregnant women (FIG. 8C left). However, the 1D NOESY spectra of the aromatic region show some differences between the preeclamptic (FIG. 8B right) and the control pregnant woman (FIG. 8C right), although these are mainly in the broad envelope of macromolecular resonances, not the metabolite signals.

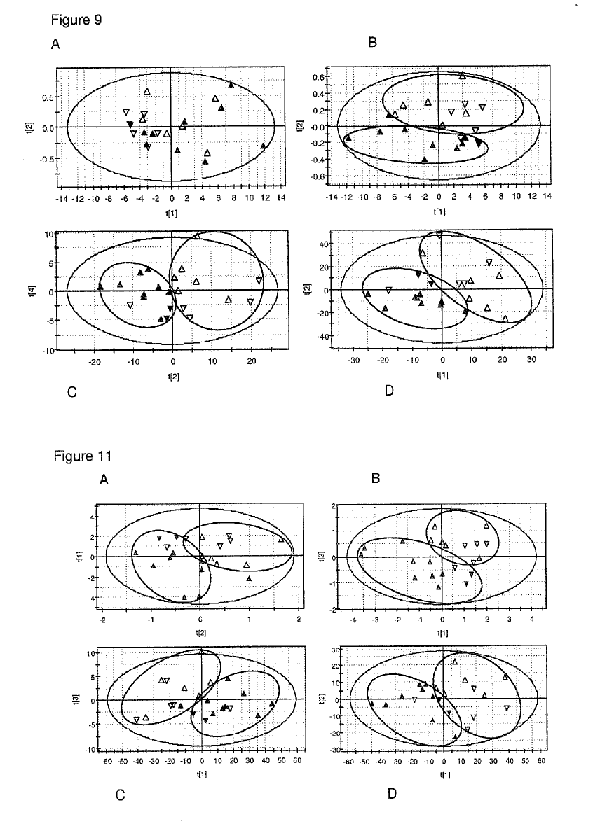

However, using the initial analysis with a bin width of 0.045 ppm across the region δ 6-9.5, chemometrics successfully distinguished between the group of women suffering from PE and those who were enjoying a healthy pregnancy (FIG. 9). FIGS. 9A and C show PCA and FIGS. 9B and D PLS-DA show scores plots of the raw data from the spin echo experiment (FIGS. 9 A and B) and first increment of the NOESY pulse sequence (FIGS. 9 C and D), when “bins” of width 0.045 ppm were used. Open triangles represent preeclamptic patients, whilst filled triangles represent normotensive control pregnant women. Inverted triangles represent those women who have already given birth to a least one baby, whilst all others represent women in their first pregnancy. This distinction is seen more clearly in the analysis of the first increment of the NOESY experiment than the spin echo experiment—whilst the groups are clearly separated in the PCA (and PLS-DA) scores plots from the 1D NOESY, this classification is only observed in the PLS-DA of the spin echo data, and is not observed in the PCA scores plots.

### EXAMPLE 5

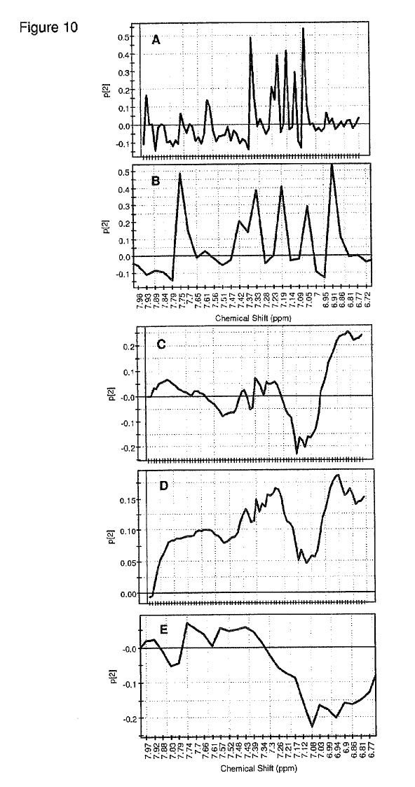

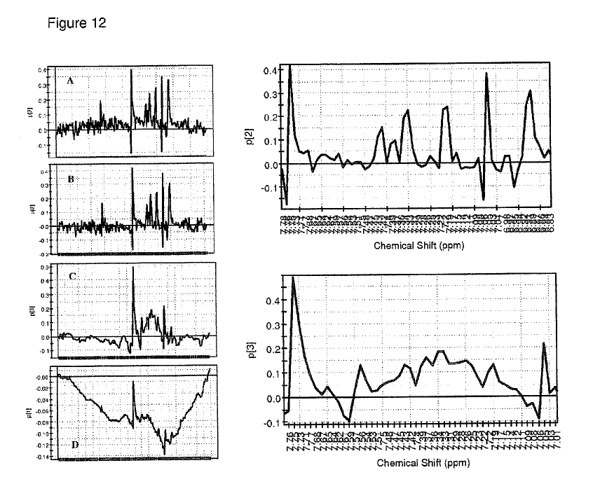

In the case of the 1D NOESY, the most important parts of the aromatic region were the same in the PCA and PLS-DA analysis, and can been seen in the loading line plots in FIG. 10. FIGS. 10 C and E show PCA and FIGS. 10 A, B and D show PLS-DA loading plots of the raw data from the spin echo experiment (FIGS. 10 A and B) and first increment of the NOESY pulse sequence (FIGS. 10 C, D and E), when “bins” of width 0.045 ppm were used. Influential regions of the spectrum shown on the above plots (expansions of FIGS. 10 A and C shown in FIGS. 10 B and E respectively) are those which cause distinction between the two groups of women and grouping on the scores plots shown in FIG. 9. These plots of the eigenvectors revealed the regions contributing most to the grouping exhibited in the scores plots shown in FIG. 9. The spectral region which appeared to be most important was δ 7.05-7.10, corresponding to the constituent histidine. This region and constituent was exposed as the most notable when both the raw integral data was used, and when the data was normalised to the signal from the reference compound TSP, at 0 ppm. The level of histidine was found to be significantly different (p=0.030 with raw data, 0.007 with normalised data) in the plasma of preeclamptic women than that of the control women. Similarly, a second influential region illustrated in the loading plots was δ 7.76-7.81, again, corresponding to a signal from histidine, although the integral values here were not significant (p=0.056 with normalised data, 0.202 with raw data). Finally, a third region, δ 7.00-7.05 was also prominent in the loading plots—this again corresponded to histidine (but also contained signals from 1-methylhistidine and 3-methylhistidine) and, again, was found to be significantly different in sufferers of PE when the data was normalised (p=0.018, and 0.196 for raw data). Other important constituents with a variation in concetration between the groups were tyrosine, found to be significantly different in different PE patients (δ 6.83-6.88, p=0.021 for normalised data and 0.194 for raw data), as well as the previously mentioned 3-methylhistidine (δ 7.00-7.05 and 7.58-7.63), histidine (δ 7.00-7.05), histidine and methyl histidine derivatives (δ 7.00-7.05 and 7.58-7.63) and phenylalanine (δ 7.36-7.45), although the differences in the concentrations of these three constituents were not found to be significant (p=0.477, 0.196 and 0.952 for raw data, and p=0.126, 0.018 (with histidine) and 0.088 for normalised data, respectively).

The spin echo experiment, although exhibiting grouping of patients on the PLS-DA scores plots, did not appear to be as successful as the first increment of the NOESY pulse sequence. From the p[2] loading plot of the PLS-DA model, the same important constituents as in the NOESY anaylsis were found to be causing the clustering of points on the scores plots—the most important region this time being δ 7.72-7.77, the constituents within this range, again, being histidine (7.73 ppm) and 1-methylhistidine (7.77 ppm). As in the NOESY analysis, the region δ 7.02-7.07, 1-methlyhistidine (7.05 ppm), was also responsible for distinction between the groups. Tyrosine, in the regions δ 6.87-6.93 (6.87 ppm) and δ 7.16-7.21 (7.17 ppm), and phenylalanine, in the regions δ 7.30-7.35 (7.33 ppm) and δ 7.38-7.44 (7.38 ppm, 7.43 ppm) were also revealed on the loadings plot as being influential. Whilst all the same constituents were picked out by the spin echo, no significant differences between the concentrations of these compounds in the PE patients' plasma and that of the control patients were found for the raw data. When the data was normalised however, all these compounds were found to be significantly different in concentration between the two groups of women (p=0.019 (his and 1-methylhis), 0.021 (his), 0.028 and 0.040 (tyr), 0.019 and 0.015 (phe)).

### EXAMPLE 6

In the second stage of the analysis, much narrower “bins” of 0.016 ppm width were used to analyse the aromatic regions of the spectra. In this part of the investigation, the reverse appears to be true for the success of the two experiments, it was the spin echo pulse sequence which most efficiently distinguished between the two groups of women (FIG. 11). FIGS. 11 A and B show PCA and FIGS. 11 C and D show PLS-DA scores plots of the raw data from the spin echo experiment (FIGS. 11 A and C) and first increment of the NOESY pulse sequence (FIGS. 11 B and D), when “bins” of width 0.016 ppm were used. Open triangles represent preeclamptic patients, whilst filled triangles represent normotensive control pregnant women. Inverted triangles represent those women who have already given birth to a least one baby, whilst all others represent women in their first pregnancy. In both the PGA and PLS-DA of the spin echo data, the women were correctly classified into two groups. FIGS. 12 A and C show PCA and FIGS. 12 B and D show PLS-DA loading plots of the raw data from the spin echo experiment (FIGS. 12 A and B) and first increment of the NOESY pulse sequence (FIGS. 12 C and D), when “bins” of width 0.016 ppm were used. Influential regions of the spectrum shown on the above plots (expansions of FIGS. 12 B and C shown top right and bottom right respectively) are those which cause distinction between the two groups of women and grouping on the scores plots shown in FIG. 11.

Loading plots (FIG. 12) from either model showed the same sections of importance of the aromatic regions of the spectra which were influential in clustering of patients on the scores plots again, histidine appeared to be the most important constituent. Two regions with the highest magnitude of eigenvectors, δ 7.742-7.758 and 7.042-7.058, both correspond to signals assigned to histidine. In this analysis, the concentrations of 1-methlyhistidine were found to be significantly higher in women suffering from PE than those enjoying a normal pregnancy (p=0.019 and 0.026)—as the binning was solely carried out over the aromatic region, no normalisation to the TSP reference peak could be performed, and so only raw data was used. Again, tyrosine, in the region δ 6.882-6.918, was found to have significantly different concentrations between the two groups of women (p=0.044)—higher levels were found in the plasma of PE patients than in that of controls, the same result occurring for another influential signal of tyrosine at δ 7.182-7.208, although this was not found to be significant. Phenylalanine (δ 7.322-7.348 and δ 7.412-7.438) was also found in parts of the aromatic region which were controlling clustering of the patients on the scores plots, though the concentrations of this constituent did not differ significantly between the two groups.

Both PCA and PLS-DA successfully differentiated between the PE and control groups for the first increment of the NOESY pulse sequence as well (FIG. 11). Again, the loading plots of either model (FIG. 12) showed the same two dominating parts of the aromatic regions as in the spin echo experiment and analysis—the regions δ 7.742-7.758 and 7.042-7.058, both corresponding to histidine. However, neither of these peaks showed any significant difference in area, and therefore in constituent concentration, between the two groups of women p=0.207 and 0.119).

In all scores plots of both the spin echo and first increment of the NOESY pulse sequence (using raw and normalised data), and using both “bin” widths, one outlier in consistently seen, this point relates to a preeclamptic woman who has been grouped with the normotensive women (FIGS. 9 and 11). On investigating the medical history of this patient, it was discovered that she was an opiate user.

### EXAMPLE 7

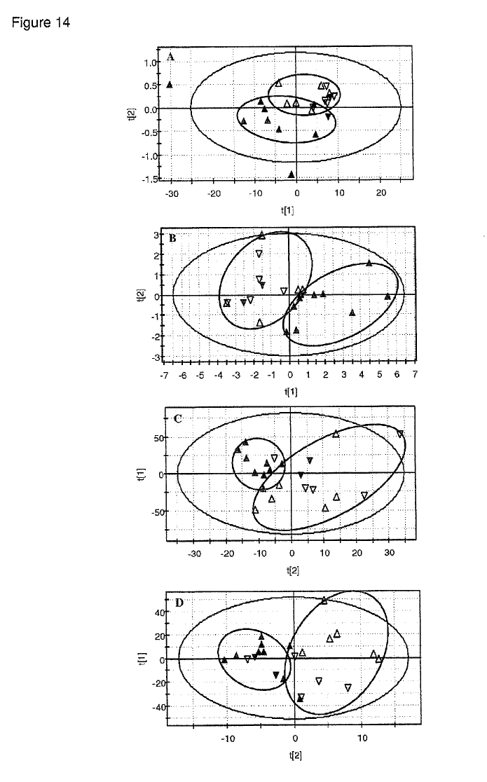

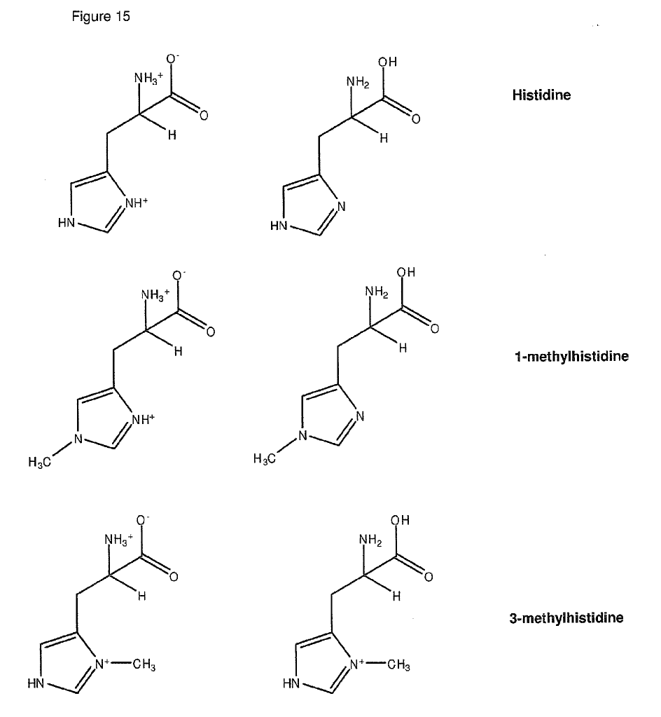

The score plots of all normalised data, when using the wider bin width of 0.045 ppm, also exhibited additional outliers. Although normalisation to the reference TSP peak was not used for the narrower 0.016 ppm “bin” analysis, this procedure was later attempted using the formate resonance at 8.45 ppm—normalisation to the formate resonance also caused additional outliers to be observed on the scores plots (FIG. 14). FIG. 14 shows scores plots of the normalised data from the spin echo experiment (FIGS. 14 A and B) and first increment of the NOESY pulse sequence (Figures C and D), when “bins” of width 0.045 ppm (Figures A and C) and then 0.016 ppm (Figures B and D) were used, showing the reduced distinction between the groups of women and an increase in the number of outliers.

The number of children to which the women had previously given birth did not seem to affect results; no within-group clustering of women pregnant with their first child, or pregnant women who had previously given birth occurred.

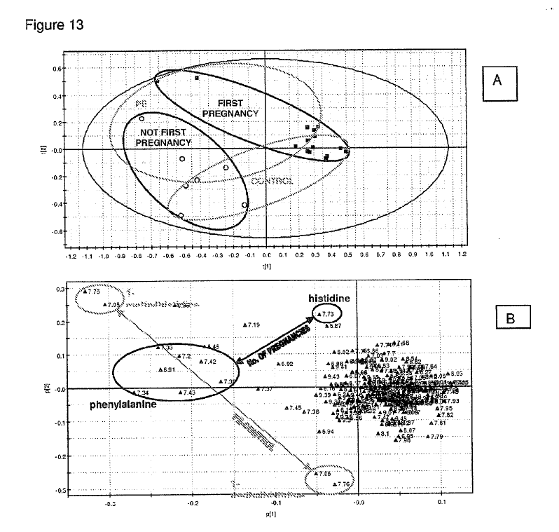

This was not the case, however, when the data from the second analysis, with a bin width of 0.016 ppm, was normalised to unit total sum of the spectral integral. This was performed as normalisation to reference compound TSP was not possible, and normalisation to formate did not seem particularly successful. The spin echo data was able to distinguish between women with PE and control pregnant women, but was also able to differentiate between those women in their first pregnancy, and those women not in their first pregnancy (FIG. 13). FIG. 13A shows a PLS-DA scores plot and corresponding loading plot (FIG. 13 B) of the data from the spin echo experiment, when “bins” of width 0.016 ppm were used, and data was normalised to unit total sum of the spectral integral. Influential regions of the spectrum, and the direction in which these occur, are shown on the loading plot (in ppm), and cause distinction between the women suffering from PE, those enjoying a normal pregnancy, women in their first pregnancy and women in their second or greater pregnancy. The models produced in the analysis of the data of the first increment of the NOESY pulse sequence were also able to group the preeclamptic women and control women accordingly, but they were not able to classify women based on the number of pregnancies each had experienced.

The corresponding loading plot for both the spin echo (FIG. 13) and 1D NOESY data, again, show that histidine or a methyl derivative thereof is responsible for the separation between the women with PE and normotensive women, but the most influential constituent in the classification of women based on the number of pregnancies was the amino acid phenylalanine, the concentration of which appeared to be significantly lower in women in their first pregnancy than in those who had given birth to one or more children before (p=0.005, 0.004, 0.017 and 0.003 for four regions of the spectrum assigned to phe).

### EXAMPLE 8

Table 1 below shows the highest and lowest values obtained from integrals of the peaks in the aromatic region of tyrosine signals at 6.89 and 7.19 ppm and for histidine at 7.05 and 7.75 ppm of the spin echo data for 21 of the total 22 samples of the control and PE groups of women. A single patient was excluded from the analysis (patient 1405, an opiate user suffering from preeclampsia, who was incorrectly classified as a control pregnant patient).

TABLE 1

| | | Highest Value | | Lowest Value | |
|---|---|---|---|---|---|
| Signal | | (Absolute integral) | | (Absolute Integral) | |
| (ppm) | Molecule | PE | Control | PE | Control |
| 6.89 | tyrosine | 0.2470 | 0.1750 | 0.0700 | 0.0330 |
| 7.05 | histidine | 0.3920 | 0.2800 | 0.1170 | 0.0560 |
| 7.19 | tyrosine | 0.1880 | 0.1530 | 0.0410 | 0.0230 |
| 7.75 | histidine | 0.4460 | 0.3840 | 0.1760 | 0.0500 |

Table 2 below shows the actual percentage increase from control to PE of the median absolute integral of tyrosine signals at 6.89 and 7.19 ppm and for histidine at 7.05 and 7.75 ppm of the spin echo data for 21 of the 22 samples of the control and PE groups of women.

TABLE 2

| | | |
|---|---|---|
| | Median | Percentage |
| Signal | (Absolute Integral) | increase from |

| (ppm) | Molecule | PE | Control | control to PE |
|---|---|---|---|---|
| 6.89 | tyrosine | 0.1485 | 0.0960 | 55 |
| 7.05 | histidine | 0.1985 | 0.1030 | 93 |
| 7.19 | tyrosine | 0.1195 | 0.0720 | 66 |
| 7.75 | histidine | 0.2840 | 0.1550 | 83 |

Table 3 below shows the actual percentage increase from control to PE of the mean absolute integral of tyrosine signals at 6.89 and 7.19 ppm and for histidine at 7.05 and 7.75 ppm of the spin echo data for 21 of the 22 samples of the control and PE groups of women.

TABLE 3

| | | |
|---|---|---|
| | Median | Percentage |
| Signal | (Absolute Integral) | increase from |

| (ppm) | Molecule | PE | Control | control to PE |
|---|---|---|---|---|
| 6.89 | tyrosine | 0.1559 | 0.0861 | 81 |
| 7.05 | histidine | 0.2122 | 0.1255 | 69 |
| 7.19 | tyrosine | 0.1137 | 0.0824 | 38 |
| 7.75 | histidine | 0.2806 | 0.1685 | 67 |

The results show that although the percentage increases in the mean and median are variable for tyrosine signals (median=55-66% and mean=38-81%), they are quite consistent for the histidine peaks (median=83-93% and mean=67-69%).

### EXAMPLE 9

Evidently, the narrower the bin, the more suitable the spin echo experiment becomes. The combination of the application of the narrower “bin” width of 0.016 ppm and the spin echo experiment seems to be the most efficient and conclusive method of investigating the aromatic biomarkers of preeclampsia. Whilst the results of the analysis of the first increment of the NOESY pulse sequence support those of the spin echo, they do not show evidence of any significant differences in constituent concentrations. Alternatively, using the “traditional” bin width of 0.045 ppm, the 1D NOESY experiment surpasses that of the spin echo, where not only excellent distinction is made between the two groups of women, but significant differences are seen in constituent concentrations of the plasma from PE and control patients in raw and normalised data, a characteristic only exhibited in the normalised data of the spin echo.

Equally, it could be argued that the “bin” width and experiment are not as important as is first believed, as the same aromatic biomarker has undoubtedly dominated all analyses throughout the investigation; that is 1-methylhistidine. Whilst this definite biomarker has certainly been most responsible for the distinction between the groups, other aromatic constituents, such as phenyalanine, histidine or methyl derivatives thereof and especially tyrosine, have also consistently been found to be influential in discrimination between PE patients and normotensive women throughout the whole investigation. However, analysing the way in which these concentrations differ between the two groups, the specific experiment and exact “bin” width become vital. The method employing the spin echo and the “bin” width of 0.016 ppm produces results where the concentration of 1-methlyhistidine is significantly higher in the plasma of women suffering from PE than that of the control group. The method of applying a bin width of 0.045 ppm to 1D NOESY spectra produces results where the concentration of 1-methlyhistidine is also significantly different between the plasma of PE patients and that of the control women.

In the present invention we have found that using ¹H-NMR metabonomics—the aromatic region alone clearly has the potential to be a valuable diagnostic tool in predicting the onset of preeclampsia in pregnant women.

1. A method of diagnosing preeclampsia in a pregnant individual or predicting the likelihood of developing preeclampsia in a pregnant individual, the method comprising comparing the levels of histidine or a methyl derivative thereof in a sample obtained from the pregnant individual to values obtained for pregnant women at a similar gestational period and known not to be suffering from preeclampsia wherein an increased level of histidine or a methyl derivative thereof in the sample indicates that the patient is suffering from or is likely to develop preeclampsia.

2. The method according to claim 1, wherein the methyl derivative is 1-methylhistidine or 3-methyl histidine.

3. The method according to claim 1 further comprising comparing the levels of lipids in a sample obtained from the pregnant individual to values obtained for pregnant women at a similar gestational period and known not to be suffering from preeclampsia wherein a decreased level of lipids in the sample indicates that the patient is suffering from or is likely to develop preeclampsia.

4. The method according to claim 3 wherein assessment of the levels of histidine or a methyl derivative thereof and lipids may be performed independently or in conjunction on the same or different sample obtained from the same individual.

5. The method according to claim 1 wherein the individual is human.

6. The method according to claim 1 wherein the sample is selected from the group consisting of whole blood, blood plasma, amniotic fluid, serum, urine, synovial fluid and cerebrospinal fluid.

7. The method according to claim 6, wherein the sample is blood plasma.

8. The method according to claim 6, wherein the sample is fresh or frozen.

9. The method according to claim 1 wherein the sample is obtained between weeks 12 and up to delivery.

10. The method according to claim 9, wherein the sample is taken at around 20 weeks gestation.

11. The method according to claim 1 further comprising comparing the levels of ketone bodies in a sample obtained from the pregnant individual to values obtained for pregnant women at a similar gestational period and known not to be suffering from preeclampsia, wherein a decreased level of ketone bodies compared to values obtained from pregnant women of a similar gestational period is a further indication of preeclampsia.

12. The method according to claim 11, wherein the ketone body is selected from the group consisting of 3-hydroxybutyrate (3HB), acetoacetate (AA) and α-ketoglutarate.

13. The method according to claim 12, wherein the ketone body is 3HB.

14. The method according to claim 1 wherein the sample is analysed by nuclear magnetic resonance (1H-NMR) spectroscopy.

15. The method according to claim 14, wherein in the instance of detecting the histidine, the pattern of the individual at risk of preeclampsia exhibits two regions with the highest magnitude of cigenvectors at δ 7.742-7.758 and 7.042-7.058.

16. The method according to claim 1 further comprising detecting levels of histidine or a methyl derivative thereof in a sample obtained from the pregnant individual using a detection method wherein the method of detection is selected from the group consisting of an enzymic assay, antibody detection and a separation technique.

17-19. (canceled)

20. A method of treating preeclampsia in a pregnant individual, the method comprising:
(i) detecting the presence of histidine or a methyl derivative thereof in a sample obtained from a pregnant individual and comparing the levels to values obtained for pregnant women at a similar gestational period and known not to be suffering from preeclampsia wherein an increased level of histidine or a methyl derivative thereof in the sample indicates that the pregnant individual is suffering from or is likely to develop preeclampsia;
(ii) detecting lipids in a sample obtained from said pregnant individual and comparing the levels to values obtained for pregnant women at a similar gestational period and known not to be suffering from preeclampsia wherein a decreased level of lipids in the sample indicates that the pregnant individual is suffering from, or is likely to develop, preeclampsia; and
(iii) using the results obtained from step (i) and/or (ii) to determine a clinical treatment for preeclampsia.

21. The method according to claim 20, wherein step (ii) is performed in advance of step (i).

22. The method according to claim 20, wherein depending on the severity of the condition a clinician is able to determine immediate or delayed therapy or is able to determine the aggressiveness of the therapy.

23. (canceled)

24. A method of identifying a pregnant individual at risk of developing preeclampsia, the method comprising
(i) detecting a level of histidine or a methyl derivative thereof in a sample obtained from an individual at around 15-25 weeks gestation period and comparing the levels to values obtained for pregnant women at a similar gestational period and known not to be suffering from preeclampsia wherein an increased level of histidine or a methyl derivative thereof in the sample indicates that the pregnant individual is suffering from, or is likely to develop, preeclampsia; and
(ii) detecting a level of lipids in said sample and comparing the levels to values obtained for pregnant women at a similar gestational period and known not to be suffering from preeclampsia wherein a decreased level of lipids in the sample indicates that the pregnant individual is suffering from or is likely to develop preeclampsia.

25. The method according to claim 24, wherein step (ii) is performed in advance of step (i).

26. The method according to claim 24, further comprising repeating the method over subsequent weeks of gestation so as to monitor progression of preeclampsia or to monitor efficacy of an intervening therapy administered to the individual to combat the condition.

27. (canceled)

28. A diagnostic kit comprising reagents required to determine the level of plasma lipid and histidine or a methyl derivative thereof.

29. (canceled)

30. The method of claim 1, wherein the method comprises analysing a sample obtained from the pregnant individual for the presence of histidine or a methyl derivative thereof in the sample.

31. The method of claim 24, wherein the method comprises analysing a sample obtained from the pregnant individual at around 15-25 weeks gestation period.
